(19) United States
(12) Patent Application Publication
(10) Pub. No.: US 2007/0207937 A1
(43) Pub. Date: Sep. 6, 2007

(54) GELLED COMPOSITION

(75) Inventors: Angeles Fonolla Moreno, Paris (FR); Pascale Jaeger-Paindestre, Fresnes (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/669,365

(22) Filed: Jan. 31, 2007

Related U.S. Application Data
(60) Provisional application No. 60/781,375, filed on Mar. 13, 2006.

(30) Foreign Application Priority Data
Mar. 6, 2006 (FR) 06 50774

(51) Int. Cl. A61K 8/00

(52) U.S. Cl. 510/130; 510/136

(57) ABSTRACT

Gelled composition containing at least 40% by weight of one or more oils, relative to the total weight of the composition, at least one ethoxylated glucose fatty acid ester, at least one ethoxylated polyol fatty acid ester, at least one polyol, and water, the amount of water being less than or equal to 15% by weight relative to the total weight of the composition, and the amount of water and the amount of polyol each being greater than or equal to the amount of ethoxylated glucose fatty acid ester.

### REFERENCE TO PRIOR APPLICATIONS

This application claims priority to U.S. provisional application 60/781,375 filed Mar. 13, 2006, and to French patent application 0650774 filed Mar. 6, 2006, both incorporated herein by reference.

### FIELD OF THE INVENTION

The invention relates to a gelled composition comprising oil, sugar-derived emulsifier, polyol ester, polyol and water, and to its use in, e.g., the cosmetics field, and especially for removing makeup from the skin and/or eyes.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Modern makeup technologies are increasingly innovative and highly performing, and there are more and more users of makeup products who use long-lasting products, such as non-transfer foundations, long-lasting lipsticks, waterproof mascaras or two-step mascaras (involving application first of a base and then of the mascara).

However, these types of product are more difficult to remove than conventional makeup products, and consequently there exists a need for makeup-remover products which combine high performance with practicality in use and which are kind to the skin while having good cosmetic qualities (comfort in use, softness).

Makeup-remover products which are currently proposed include the following:
conventional technologies such as makeup-remover lotions, whose effectiveness on long-lasting makeup is limited and often inadequate.
Two-phase products separately comprising an aqueous phase and an oily phase, the two phases being emulsified in one another only at the time of use, and undergoing phase separation again at rest; these products are highly performing but are liquid and therefore runny, which is not always practical, and they are not always valued for the removal of makeup from the eyes, owing to the fact that the oily phase may sometimes leave a slight oily film over the eyes.
Micellar solutions based on water and surfactants, which offer very high comfort but are not very effective with respect to waterproof mascaras.
Makeup-remover oils, which have a great presence in the Japanese market and which are extremely effective on non-transfer products but are not very effective on the more conventional products. Moreover, since these oils are liquids, their use is not very convenient, since there is a risk of the product running over the hands.

### SUMMARY OF THE INVENTION

There therefore continues to be a need to have a makeup-remover product which at the same time performs highly on all types of makeup (conventional and long-lasting), is practical to use and is comfortable.

The inventors have now found, surprisingly, that the combination of surfactants with makeup-removal oils in a composition likewise containing water and polyol allows an oily gel to be obtained which is easy to rinse off, the surfactants on the one hand allowing the formula to gel and on the other hand facilitating its removal by rinsing.

### DETAILED DESCRIPTION OF THE PREFERRED EMBODIMENTS

The present invention provides compositions containing at least 40% by weight of one or more oils, relative to the total weight of the composition, at least one ethoxylated glucose fatty acid ester, at least one ethoxylated polyol fatty acid ester, at least one polyol, and water, the amount of water being less than or equal to 15′ by weight relative to the total weight of the composition, and the amount of water and the amount of polyol each being greater than or equal to the amount of ethoxylated glucose fatty acid ester.

The compositions according to the invention are preferably transparent or translucent and preferably also gelled, and therefore easy to use, while at the same time being comfortable and allowing effective removal of any type of makeup. Furthermore, they preferably exhibit very good stability.

The transparency of the invention compositions is evident visually. It can be seen, for example, that when applied to the skin, in a thickness of 5 mm, for example, the composition is transparent; that is, it allows the skin to show through.

By “gelled” is meant the fact that the composition does not run, in other words that it has a certain viscosity. Its viscosity may range for example from 5 to 190 poises (0.5 to 19 Pa·s), preferably from 5 to 150 poises (0.5 to 15 Pa·s) and more preferably from 10 to 120 poises (1 to 12 Pa·s), this viscosity being measured using the Rheomat 180 viscometer at a shear rate of 200 s⁻¹ and at 25° C.

Moreover, the phrase “amount of water and the amount of polyol each being greater than or equal to the amount of ethoxylated glucose fatty acid ester” means that, independently of one another, the amount of water, on the one hand, and the amount of polyol, on the other, are greater than or equal to the amount of ethoxylated glucose fatty acid ester. The amount of water and the amount of polyols are each greater than or equal to the amount of ethoxylated glucose fatty acid ester.

The composition according to the invention is preferably intended for topical application and therefore preferably comprises a physiologically acceptable medium. A physiologically acceptable medium is one which is compatible with the skin, the mucosae (including the inside of the eyelids, and the lips), the nails, the ocular area and/or the keratin fibres (hair and lashes).

The compositions according to the invention comprise at least one ethoxylated glucose fatty acid ester. This ester is preferably an ethoxylated glucose stearate, and in particular is methyl glucose sesquistearate containing 20 moles of ethylene oxide (INCI name: PEG-20 methyl glucose sesquistearate), such as the product sold as Glucamate SSE-20 by Amerchol and that sold as Grillocose PSE-20 by Goldschmidt.

The amount of ethoxylated glucose fatty acid ester(s) may range, for example, from 0.2% to 15% by weight, preferably from 0.5% to 15% by weight, more preferably from 0.5% to 10% by weight, more preferably still from 1% to 10% by weight and very preferably from 2% to 5% by weight relative to the total weight of the composition.

The composition further comprises one or more other surfactants which are nonionic and are preferably selected from ethoxylated polyol fatty acid esters and, more preferably, from ethoxylated glyceryl fatty acid esters. Ethoxylated glyceryl fatty acid esters (identified by their INCI names) include, for example, PEG-20 glyceryl triisostearate such as that sold by Nihon Emulsion as Emalex GWIS-305 and Emalex GWIS-320EX, and PEG-7 glyceryl cocoate such as that sold by Cognis as Cetiol HE, and mixtures thereof. According to one preferred embodiment of the invention the composition comprises PEG-20 glyceryl triisostearate.

The amount of ethoxylated polyol fatty acid ester(s) may range, for example, from 0.5% to 15% by weight, preferably from 0.5% to 10% by weight, more preferably from 1% to 10% by weight, more preferably still from 1% to 5% by weight and very preferably from 2% to 4% by weight relative to the total weight of the composition.

The composition according to the invention may optionally contain one or more other surfactants, particularly nonionic surfactants, but preferably only insofar as the presence of these surfactants is not detrimental to the transparency and comfort (harmlessness) of the composition.

The composition according to the invention contains a positive amount of water which is less than or equal to 15% by weight relative to the total weight of the composition. The amount may range, for example, from 0.5% to 15% by weight, preferably from 0.6% to 15% by weight, more preferably from 0.6% to 13% by weight, more preferably still from 1% to 10% by weight and very preferably from 3% to 6% by weight relative to the total weight of the composition.

The water used in the composition of the invention may be pure demineralized water or else mineral water and/or thermal spring water and/or sea water; in other words, the water of the composition may be composed in part or in total of a water selected from mineral waters, thermal spring waters, sea waters and mixtures thereof. Generally speaking, a mineral water is suitable for consumption, which is not always the case with a thermal spring water. Each of these waters contains, among other constituents, solubilized minerals and/or trace elements. The use of these waters for specific treatment purposes is known, depending on the specific minerals and trace elements which they contain, such treatments including the moisturizing and desensitizing of the skin or the treatment of certain dermatoses. The terms “mineral waters” or “thermal spring waters” will be used to denote not only natural mineral or thermal spring waters but also natural mineral or thermal spring waters which have been enriched with additional mineral constituents and/or trace elements, and also aqueous mineral solutions and/or aqueous solutions containing trace elements that have been prepared from purified water (demineralized or distilled water).

A natural thermal spring or mineral water used according to the invention may be selected, for example, from Vittel water, Vichy basin water, Uriage water, La Roche Posay water, Bourboule water, Enghien-les-Bains water, Saint Gervais-les-Bains water, Néris-les-Bains water, Allevar-les-Bains water, Digne water, Maizières water, Neyrac-les-Bains water, Lons-le-Saunier water, Eaux Bonnes water, Rochefort water, Saint Christau water, Fumades water, Tercis-les-bains water and Avene water.

The composition of the invention comprises one or more polyols. Polyols which can be used in the composition according to the invention include, in particular, glycerol, propylene glycol, butylene glycol, hexylene glycol, polyethylene glycols such as PEG-8, dipropylene glycol and mixtures thereof. According to one preferred embodiment of the invention the polyol is glycerol, which gives better transparency to the composition than the other polyols. The glycerol can be admixed with other polyols, provided that the qualities of the composition, and particularly the transparency, are maintained.

The amount of polyol(s) may range for example from 0.5% to 15% by weight, preferably from 0.5% to 10% by weight, more preferably from 1% to 10% by weight, more preferably still from 2% to 10% by weight and very preferably from 2% to 8% by weight relative to the total weight of the composition.

The composition according to the invention comprises at least one oil, in particular a cosmetic oil.

An oil is a fatty substance which is liquid at ambient temperature (25° C.).

The amounts of oils is at least 40% by weight relative to the total weight of the composition, preferably at least 50% by weight, more preferably at least 60% by weight and more preferably still at least 70% by weight of oils, relative to the total weight of the composition. The amount of oils may range in particular from 40% to 98% by weight, preferably from 50% to 98%, more preferably from 60% to 95% by weight and more preferably still from 70% to 90% by weight relative to the total weight of the composition.

Oils which can be used more particularly in the composition of the invention include, for example:
hydrocarbon oils of animal origin, such as perhydrosqualene (or squalane);
hydrocarbon oils of plant origin, such as liquid triglycerides of fatty acids containing 4 to 10 carbon atoms, such as triglycerides of heptanoic or octanoic acids or else, for example, sunflower oil, corn oil, soya oil, marrow oil, grapeseed oil, sesame oil, hazelnut oil, apricot oil, macadamia oil, arara oil, castor oil, avocado oil, caprylic/capric acid triglycerides such as those sold by Stearineries Dubois or those sold as Miglyol 810, 812 and 818 by Dynamit Nobel, jojoba oil and shea butter oil;
fatty acid esters and fatty alcohol ethers, such as oils of formulae R¹COOR² and R¹OR² in which R¹ represents the residue of a fatty acid containing 8 to 29 carbon atoms and R² represents a branched or unbranched hydrocarbon chain containing 3 to 30 carbon atoms, such as, for example, purcellin oil, isononyl isononanoate, isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate, and fatty alcohol heptanoates, octanoates and decanoates; polyol esters, such as propylene glycol dioctanoate, neopentyl glycol diheptanoate and diethylene glycol diisononanoate; pentaerythritol esters such as pentaerythrityl tetraisostearate; lipophilic derivatives of amino acids, such as isopropyl lauroyl sarcosinate, sold as Eldew SL 205 by Ajinomoto;
linear or branched hydrocarbons of mineral or synthetic origin, such as mineral oils (mixture of petroleum-derived hydrocarbon oils; INCI name: Mineral oil), volatile or non-volatile liquid paraffins and their derivatives, petroleum jelly, polydecenes, isohexadecane, isododecane, hydrogenated isoparaffin such as Parléam® oil, sold by NOF Corporation (INCI name: Hydrogenated Polyisobutene);
silicone oils, such as volatile or non-volatile polydimethylsiloxanes (PDMS), containing a linear or cyclic silicone chain, which are liquid or pasty at ambient temperature, especially cyclopolydimethyl-siloxanes (cyclomethicones) such as cyclopentasiloxane and cyclohexadimethylsiloxane; polydimethylsiloxanes containing alkyl, alkoxy or phenyl groups pendently or at the silicone chain end, said groups having 2 to 24 carbon atoms; phenyl silicones such as phenyl-trimethicones, phenyldimethicones, phenyltrimethyl-siloxydiphenylsiloxanes, diphenyldimethicones, diphenyldimethyldiphenyltrisiloxanes, 2-phenylethyl trimethylsiloxysilicates, and polymethylphenyl-siloxanes;
fluoro oils such as partially hydrocarbon-based and/or silicone-based oils, such as those described in document JP-A-2-295912;
C₁₂-C₁₅ fatty alcohol benzoates, such as those sold as Finsolv TN by Finetex;
the dicaprylyl carbonate sold as Cetiol CC by Cognis;
the dicaprylyl ether sold as Cetiol OE by Cognis;
and mixtures thereof.

The composition preferably contains at least one polar oil, which may be selected in particular from oils of plant origin, fatty acid esters and ethers, and mixtures thereof.

When the composition of the invention is used as a makeup-remover composition, it preferably contains at least one makeup-remover oil, which may be admixed with one or more other oils, which may or may not be makeup-remover oils.

The makeup-remover oils may be selected in particular from the branched hydrocarbons described above, the fatty acid esters described above, and mixtures thereof.

The fatty acid esters are preferably selected from esters obtained from a linear or branched-chain alcohol having 1 to 17 carbon atoms or a linear or branched-chain fatty acid having 3 to 18 and preferably 12 to 17 carbon atoms.

Fatty acid esters which can be used as makeup remover oils include more particularly ethylhexyl palmitate, ethylhexyl stearate, isopropyl myristate, isopropyl palmitate, isobutyl palmitate, pentaerythritol caprate/caprylate, cetearyl isononanoate, isodecyl isononanoate, isononyl isononanoate, isotridecyl isononanoate, and 2-ethylhexyl caprate/caprylate.

According to one particular embodiment of the invention the composition may further comprise at least one oil selected from branched hydrocarbons of mineral or synthetic origin, such as, in particular, isoparaffin, isohexadecane, isododecane and hydrogenated polyisobutene such as Parléam® oil.

It can also be advantageous for the composition according to the invention to contain a short-chain ester such as dicaprylyl carbonate or a short-chain ether such as dicaprylyl ether, in order to enhance the makeup-remover efficacy of the composition.

The compositions of the invention may further comprise adjuvants such as those that are commonplace in the cosmetics field, such as antioxidants, perfumes, perfume peptizers, colorants and hydrophilic or lipophilic actives. The nature of the adjuvants, and their amounts, should be such that they do not overly negatively modify the properties of the composition according to the invention. The amounts of these adjuvants may be those conventionally used, and are for example from 0.001% to 10% of the total weight of the composition.

Actives which can be used in the composition of the invention include, for example, soothing agents such as allantoin and bisabolol; floral waters such as lime water or cornflower water; glycyrrhetinic acid and its salts; antibacterials such as octopirox, triclosan and triclocarban; essential oils; vitamins such as, for example, retinol (vitamin A), ascorbic acid (vitamin C), tocopherol (vitamin E), niacinamide (vitamin PP or B3), panthenol (vitamin B5) and their derivatives, such as, for example, the esters of these vitamins (palmitate, acetate, propionate), magnesium ascorbyl phosphate, glycosylated vitamin C or glucopyranosyl-ascorbic acid (ascorbyl glucoside); coenzymes such as coenzyme Q10 or ubiquinone and coenzyme R or biotin; protein hydrolysates; plant extracts and particularly plankton extracts; and mixtures thereof.

The person skilled in the art would of course take care to select any additive or additives to be added to the composition according to the invention in such a way that the advantageous properties intrinsically attached to the composition in accordance with the invention are not, or not substantially, adversely affected by the intended addition.

The compositions of the invention may be used in any application. They are particularly suited to the removal of makeup from the skin and/or ocular area and/or lips.

Accordingly the invention further provides for the cosmetic use of the composition as defined above for removing makeup from the skin and/or ocular area and/or lips.

The mode of use for removing makeup comprises applying the composition to the skin and, optionally, in subsequently rinsing the skin.

The examples below are given to illustrate the invention and do not limit it. All of the amounts are given as percentages by weight relative to the total weight of the composition. The names of compounds are indicated, respectively, as chemical names or as INCI names.

### INVENTIVE EXAMPLE 1

### Makeup Remover Oil

| Phase I | |
|---|---|
| Water | 3.5% |
| Glycerol | 4.5% |
| Phase II | |
| PEG-20 methyl glucose sesquistearate | 2% |
| PEG-20 glyceryl triisostearate | 1% |
| Caprylic/capric triglyceride | qsp 100% |
| Isohexadecane | 20% |
| Ethylhexyl stearate | 20% |

Procedure: Phase II was heated to 45° C. and then, when phase II was homogeneous, it was cooled to 25° C. and was subsequently introduced into phase I with stirring using a turbine stirrer.

This gave a gelled transparent composition which did not run and had a good stability. This composition proved very comfortable in use, and exhibited highly effective makeup removal.

This composition can be used, for example, by direct application to the face with the fingers or a cotton pad, followed by rinsing.

### INVENTIVE EXAMPLE 2

### Makeup Remover Oil

| Phase I | |
|---|---|
| Water | 5% |
| Glycerol | 10% |
| Phase II | |
| PEG-20 methyl glucose sesquistearate | 3.5% |
| PEG-20 glyceryl triisostearate | 3% |
| Dicaprylyl carbonate (Cetiol CC) | 10% |
| Isododecane | qsp 100% |
| Ethylhexyl palmitate | 20% |

Procedure: identical to that of Example 1.

This gave a gelled transparent composition which did not run and had a good stability. This composition proved very comfortable in use, and exhibited highly effective makeup removal.

Its mode of use is the same as for Example 1.

### INVENTIVE EXAMPLE 3

| Phase I | |
|---|---|
| Water | 3% |
| Glycerol | 5.5% |
| Phase II | |
| PEG-20 methyl glucose sesquistearate | 2.5% |
| PEG-20 glyceryl triisostearate | 4% |
| Caprylic/capric triglycerides | qsp 100% |
| Dicaprylyl ether | 3% |
| Isohexadecane | 15% |
| Ethylhexyl stearate | 15% |
| Tocopherol acetate | 0.1% |

Procedure: identical to that of Example 1.

Its mode of use is the same as for Example 1.

The composition obtained is gelled and exhibits very high stability.

### COMPARATIVE EXAMPLE 1

| Phase I | |
|---|---|
| Water | 0.5% |
| Glycerol | 5% |
| Phase II | |
| PEG-20 methyl glucose sesquistearate | 2% |
| PEG-20 glyceryl triisostearate | 4% |
| Isononyl isononanoate | qsp 100% |
| Isohexadecane | 40% |
| Ethylhexyl stearate | 30% |

Procedure: identical to that of Example 1.

A composition was obtained which had a poor stability, with phase separation observed after 48 hours (separation of the oil and the water, with release of the oil), rather than a stable transparent gel being obtained.

When the amount of water is less than that of the glucose-derived surfactant (PEG-20 methyl glucose sesquistearate), this comparative example shows that the aim of the invention is not achieved. The result is the same when the amount of glycerol is less than the amount of said surfactant.

### COMPARATIVE EXAMPLE 2

| Phase I | |
|---|---|
| Water | 3% |
| Glycerol | 5.5% |
| Phase II | |
| PEG-20 methyl glucose sesquistearate | 2.5% |
| Caprylic/capric triglycerides | qsp 100% |
| Dicaprylyl ether | 3% |
| Isohexadecane | 15% |
| Ethylhexyl stearate | 15% |
| Tocopherol acetate | 0.1% |

Procedure: identical to that of Example 1.

A composition was obtained which had a poor stability, since it was impossible to incorporate all of the oil and since phase separation was observed at the preparation stage.

This comparative example shows that, when the composition contains no ethoxylated polyol fatty acid ester, the resulting composition is not stable and cannot be produced, whereas in the presence of ethoxylated polyol fatty acid ester (Inventive Example 3) it is possible to produce the composition and it is stable.

The above written description of the invention provides a manner and process of making and using it such that any person skilled in this art is enabled to make and use the same, this enablement being provided in particular for the subject matter of the appended claims, which make up a part of the original description and including a composition containing at least 40% by weight of one or more oils, relative to the total weight of the composition, at least one ethoxylated glucose fatty acid ester, at least one ethoxylated polyol fatty acid ester, at least one polyol, and water, the amount of water being less than or equal to 15% by weight relative to the total weight of the composition, and the amount of water and the amount of polyol each being greater than or equal to the amount of ethoxylated glucose fatty acid ester. As used herein, the ethoxylated polyol fatty acid ester does not include the ethoxylated glucose fatty acid ester, both of which are different from the oils used herein.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition comprising:
at least 40% by weight relative to the total weight of the composition of one or more oils,
at least one ethoxylated glucose fatty acid ester,
at least one ethoxylated polyol fatty acid ester,
at least one polyol, and
water,
the amount of water being less than or equal to 15% by weight relative to the total weight of the composition, and the amount of water and the amount of polyol each being greater than or equal to the amount of ethoxylated glucose fatty acid ester.

2. The composition according to claim 1, wherein the ethoxylated glucose fatty acid ester is methyl glucose sesquistearate containing 20 moles of ethylene oxide.

3. The composition according to claim 1, wherein the amount of ethoxylated glucose fatty acid ester(s) ranges from 0.5% to 15% by weight relative to the total weight of the composition.

4. The composition according to claim 1, comprising an ethoxylated glyceryl fatty acid ester.

5. The composition according to claim 4, wherein the ethoxylated glyceryl fatty acid ester is PEG-20 glyceryl triisostearate.

6. The composition according to claim 1, wherein the amount of the ethoxylated polyol fatty acid ester(s) ranges from 0.5% to 15% by weight relative to the total weight of the composition.

7. The composition according to claim 1, wherein the polyol is glycerol.

8. The composition according to claim 1, wherein the amount of polyol(s) ranges from 0.5% to 15% by weight relative to the total weight of the composition.

9. The composition according to claim 1, wherein the amount of oils ranges from 40% to 98% by weight relative to the total weight of the composition.

10. The composition according to claim 1, comprising at least one polar oil.

11. The composition according to claim 1, comprising at least one oil selected from oils of plant origin, fatty acid esters and fatty alcohol ethers, and mixtures thereof.

12. The composition according to claim 1, comprising methyl glucose sesquistearate containing 20 moles of ethylene oxide, PEG-20 glyceryl triisostearate, and glycerol.

13. The composition according to claim 1, having a viscosity of 5 to 190 poise as measured using a Rheomat 180 viscometer at a shear rate of 200 s⁻¹ at 25° C.

14. The composition according to claim 1, having a viscosity of 10 to 120 poises as measured using a Rheomat 180 viscometer at a shear rate of 200 s⁻¹ at 25° C.

15. The composition according to claim 12, having a viscosity of 5 to 190 poises as measured using a Rheomat 180 viscometer at a shear rate of 200 s⁻¹ at 25° C.

16. The composition according to claim 13, which, when applied to the skin in a thickness of 5 mm allows the skin to show through.

17. The composition according to claim 14, which, when applied to the skin in a thickness of 5 mm allows the skin to show through.

18. The composition according to claim 15, which, when applied to the skin in a thickness of 5 mm allows the skin to show through.

19. A method, comprising applying the composition of claim 1 to the skin and/or ocular area and/or lips.

20. A method, comprising applying the composition of claim 18 to the skin and/or ocular area and/or lips.
